(19) United States
(12) Patent Application Publication
(10) Pub. No.: US 2013/0261131 A1
(43) Pub. Date: Oct. 3, 2013

(54) PHARMACEUTICAL COMPOSITIONS AND MENTODS FOR TREATMENT OF FUNGAL INFECTIONS

(71) Applicant: Nian Wu, North Brunswick, NJ (US)

(72) Inventor: Nian Wu, North Brunswick, NJ (US)

(21) Appl. No.: 13/853,092

(22) Filed: Mar. 29, 2013

Related U.S. Application Data
(60) Provisional application No. 61/617,766, filed on Mar. 30, 2012.

(51) Int. Cl. A61K 31/506; A61K 31/496

(52) U.S. Cl. CPC A61K 31/506; A61K 31/496
USPC 514/254.07; 514/256

(57) ABSTRACT

Pharmaceutical compositions and methods for treating skin or mucus fungal infections of feet, nails, groin, hands, tongue and other locations are disclosed herein. The pharmaceutical composition disclosed herein comprises at least one triazole agent. Methods of treating tinea pedis, tinea manuum, tinea unguium, tinea cruris, tinea corpora, tinea versicolor, candidiasis, aspergillosis, onychomycosis, coccidiodomycosis, cryptococcal meningitis, histoplasmosis, hoof thrush, hoof rot, tongue and mouth lesions, seborrheic dermatitis, and combinations thereof, are also presently disclosed.

This application claims priority to the provisional patent application Ser. No. 61/617,766, entitled “Topic Triazole Formulations for Fungal Infections” filed in the U.S. Patent and Trademark Office on Mar. 30, 2012, by Nian Wu.

### FIELD OF THE INVENTION

The present invention relates to formulations useful for treating skin or oral fungal infection of feet, nails, groin, hands, tongue and other locations. In particular, the present invention relates to topical formulations containing triazole agents for the treatment of tinea pedis, tinea manuum, tinea unguium, tinea versicolor, tinea cruris, tinea corpora, candidiasis, onychomycosis, aspergillosis, coccidioidomycosis, cryptococcal meningitis, histoplasmosis, hoof thrush, hoof rot, tongue and mouth lesions, seborrheic dermatitis, other fungal infections, or combinations thereof.

### BACKGROUND OF THE INVENTION

Itraconazole, voriconazole and posaconazole are broad-spectrum triazole antifungal compounds. The antifungal mode of action of all three compounds may be by selective inhibition of the α-demethylase P450 cytochrome system. The advantages to these three compounds may be that there is a substantial safety profile in hundred million patients, representing hundreds of millions of doses. Additionally, it is a second generation drug in a class that has proven effectiveness but has demonstrated better efficacy. Similarly equaconazole [WO2,010,027,496] is the third generation triazole, currently under clinical development, that present improved pharmacology and safety profiles.

In clinically relevant animal models of fungal infections, posaconazole and equaconazole may be very active against both pulmonary (compromised) and systemic (normal) A. fumigatus, A. flavus and C. albicans (including Fluconazole-resistant) infections in mice, when given therapeutically or prophylactically. Posaconazole and equaconazole also may be very active against vaginal infections in hamsters when given orally or topically. Posaconazole and equaconazole are active against T. mentagrophytes infections in guinea pigs when given orally or topically.

A pharmacophore model of azole antifungals was proposed initially using miconazole as an example [Talele, T. T & Kulkarni V. M., J. Chem. Inf. Comput. Sci. 1999, 39, 204-210]. A similar model may be applied to itraconazole, where the pharmacophore consists of a trizole ring and a halogenated phenyl ring, both of which are attached to C5 of a 1,3-dioxalane. In both cases the pharmacophores interact with a hydrophobic cavity in the active site of P450_{14DM}. It has been suggested that hydrogen bonds formed between the OH group of substrate and carbonyl and amino groups of the main chain and hydroxyl group of the side chain of P450_{14DM} may be essential for orienting the substrate to the correct direction in the active site [Aoyama, Y., Yoshida, Y., Sonoda, Y., Sato, Y., Biochim. Biophys. Acta, 1989, 1006, 209-213; Aoyama, Y., Yoshida, Y., Sonoda, Y., Sato Y., Biochim. Biophys. Acta, 1989, 1001, 196-200; Aoyama, Y., Yoshida, Y., Sonoda, Y., Sato Y., Biochim. Biophys. Acta, 1991, 1081, 262-266; Aoyama, Y., Yoshida, Y., Sonoda, Y. & Sato, Y., Biochim. Biophys. Acta, 1992, 1122, 251-255]. Therefore hydroxyl groups in these antifungals may have been the essential structures for interacting with the fungal P450 _{14DM} protein. A good geometrical fit of pharmacophores and values of the energy difference between the resulting bioactive conformation and the minimum energy for the conformation argued for a reasonable common conformation framework.

A study using a three-dimensional molecular model of P450_{14DM} from Saccharomyces cerevisiae based on homology with P450BM3 was reported [Lewis, D. F. V., Wiseman, A. & Tarbit, M. H., J. Enzyme Inhibit. 1999, 14, 175-192]. The halogenated phenyl ring of ketoconazole was proposed to occupy the same hydrophobic cavity as the 17-alkyl side chain of lanosterol in the model. S378 may have been identified to interact with the 3-hydroxy group of the substrate, and the 17-alkyl side chain may have been deep in the same hydrophobic cavity.

Most azole antifungals contain a halogenated phenyl group which has a similar docking mode in the active site of the fungal P450_{14DM} protein. The active site residues interacting with the inhibitors may be the same as those interacting with the substrate, i.e., the halogenated phenyl group of the inhibitors is interactive with the same hydrophobic binding cleft as the 17-alkyl chain of substrate. Since the hydrophobic cleft is narrow, the space adjacent to the phenyl group is limited. Thus, bulky substituents larger than a chlorine atom may produce significant steric clashes and lower binding affinity [Klopman, G. & Ptchelintsev, D., J. Comput.-Aided Mol. Des. 1993, 7, 349-362; Asai, K., Tsuchimori, N., Okonogi, K., Perfect, J. R., Gotoh, O. & Yoshida, Y., Antimicrob. Agents Chemother. 1999, 43, 1163-1169].

Although the side chains of itraconazole, ketoconazole, equaconazole (under clinical development), and posaconazole may be very long, while the side chains of fluconazole, isavuconazole (currently in Phase III clinical trials) and voriconazole may be rather short, all of them showed high antifungal activities. The reason may be that all of them have the same pharmacophores and the spatial orientations of the pharmacophores are very similar. Even though the side chains of these inhibitors may not be the determinants for the anti-fungal activity, they may play an important role in adjusting the physicochemical properties of the whole molecule to avoid some dissatisfying side effects and/or improve their pharmacokinetic and pharmacodynamic behavior. The side chains of itraconazole, ketoconazole, equaconazole, and posaconazole may be too long to be accommodated in the active site. However, the long side chains of the inhibitors may interact with the residues in the substrate access channel. Especially for itraconazole and posaconazole, the terminal alkyl group of the side chain may reach the entrance of the substrate access channel and may interact with the hydrophobic residues [Talele, T. T. & Kulkarni V. M., J. Chem. Inf. Comput. Sci. 1999, 39, 204-210].

Skin mycosis is thought to be the world's most common dermatophytosis. For example, 70% of the population may become infected with tinea pedis at some time. [http://www.bhchp.org/BHCHP%20Manual/pdf_files/Part1_PDF/TineaPedis.pdf accessed on Feb. 19, 2011]. With the limitations of current anti-fungal agents, it is desirable to develop new and improved agents and compositions for treatment.

### BRIEF SUMMARY OF THE INVENTION

Topical Formulations of triazole antifungal agents are disclosed. These triazoles provide clinical treatments for skin and oral mycoses.

### DETAILED DESCRIPTION OF THE INVENTION

Embodiments of the present invention are described herein in the context of novel application of triazole antifungal agents. Those of ordinary skill in the art will realize that the following detailed description of the present invention is illustrative only and is not intended to be in any way limiting. Reference will now be made in detail to implementations of the present invention.

In the present invention, topical antifungal compositions for treating fungal infections or mycoses include triazole antifungal agents are described. The composition may contain a PEG-lipid as the pharmaceutical carrier.

In a preferred embodiment, the antifungal agent is a triazole type agent such as Voriconazole or Posaconazole or Equaconazole.

Clinically oral itraconazole has been approved more effective and safer than terbinafine and fluconazole [De Keyser P, De Backer M, Massart D L, Westelinck K J. Br J Dermatol. 1994, 130 Suppl 43:22-5; Gupta A K, Ginter G. Pediatr Dermatol. 2001, 8, 519-22; Wisuthsarewong W, Chaiprasert A. J Med Assoc Thai. 2005, 88 Suppl 8:S72-9]. However there may not be triazole topical formulation available in clinical use.

In comparison with itraconazole, voriconazole and posaconazole may be significantly more potent than itraconazole [Munayyer, H., K. J. Shaw, R. S. Hare, B. Salisbury, L. Heimark, B. Pramanik, & J. R. Greene. 1996. “SCH 56592 is a potent inhibitor of sterol C14 demethylation in fungi.” 36^{th} Interscience Conference on Antimicrobial Agents and Chemotherapy; Nguyen M. H. and Yu, C. Y. Antimicrob Agents Chemother. 1998, 42, 471-472,].

The presently claimed invention may also include equaconazoles, which may have extended pharmacophores and/or improved physicochemical properties of antifungal activity and may be useful in the medical treatment of fungal infections in humans and animals.

The antifungal agents of the presently claimed invention may be typically formulated with one or more pharmaceutically acceptable carriers that are known in the art. In a preferred mode, the agents may be formulated into liposomes as described in U.S. Pat. No. 8,304,565, which is hereby incorporated by reference. Preferred diacylglycerol-polyethyleneglycol (DAG-PEG) lipids include PEG-12-GDO, PEG-12-GDM, PEG-12- GDLO, PEG-12-GDP, PEG-12-GDO, PEG-12-GDM or any combination thereof. GDO means glycerol dioleate, GDM means glycerol dimyristate, GDLO means glycerol dilinoleate, GDL means glycerol dilaurate, and GDP means glycerol dipalmitate. The numeral after the PEG means the number of subunits in the PEG chain. For example, PEG-12 refers to a PEG chain having 12 subunits.

In another aspect the present disclosure, the antifungal agents may be formulated with lipid-carbohydrate-polymers (LCP) described in PCT Publication No. WO2012109112, which is hereby incorporated by reference. The lipid-carbohydrate-polymers may have a variety of amino acid or chemical linkages between the central backbone and the carriers, may be combined with other commercial available lipid-polymers for the topic formulations.

In a further aspect of the present disclosure, a new route for the effective prevention and treatment of fungal infections in mammals may be provides. Additionally, the present disclosure may provide pharmaceutical formulations for such prevention and treatment. A method of treatment of topical and oral fungal infections in mammals may also be provided in the present disclosure.

In another aspect, the present disclosure may provide a pharmaceutical composition for treating or preventing fungal infection comprising an antifungally effective amount of a triazole together with a pharmaceutically acceptable carrier therefore. The pharmaceutically acceptable carrier may be consisting of a DAG-PEG or a LCP.

In another aspect the disclosure, a method of treating and/or preventing a fungal infection in a mammal may comprise administering an antifungally effective amount of a tiazole sufficient for such treating or preventing. The method may employ a means selected from the carrier consisting of lipids. The pharmaceutically acceptable carrier may be a mixture of 2 or more lipid or polymer modified lipids.

While the preferable compounds in the present disclosure may be thermally stable, as well as physically and chemically compatible with commonly used pharmaceutical excipients, they are water insoluble triazole compounds which may result in low and variable permeability in animals if administered without a proper formulation. Lipid based formulations using DAG-PEG or a LCP are disclosed herein to enhance the permeability and to reduce required amounts of lipids or co-solvents which may cause possible hypersensitivity or irritation by those formulations containing high concentrations of polymers and organic solvents. Therefore, in another aspect the present disclosure may include a method of making a pharmaceutical composition for treating or preventing a fungal infective comprising combining a compound shown in chemical structures 1-3 with a DAG-PEG or a LCP and an aqueous solution to compose a lipid based formula.

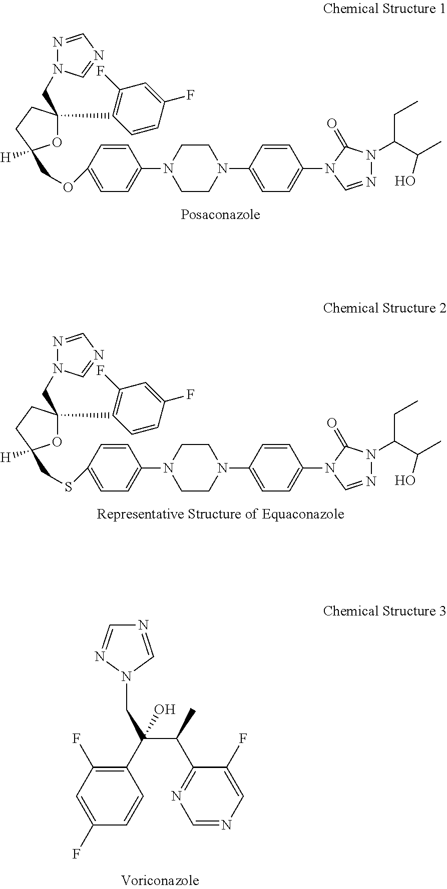

Embodiments of the present invention are described herein in the context of preparation of pharmaceutical compositions including a triazole for external or intra-mouth administrations. The treatment or prevention of the fungal infection afflicted with dermal tissue, subdermal tissue, mucosal membranes, or combinations thereof.

The approximate preferable compositions for formulated drug products are generally described herein, though different drugs typically have differing optimal formulations.

For aerosol preparations, a preferable concentration of drug is 0.01 to 2%. More preferable is 0.05 to 2%. Most preferable is 0.1 to 0.5%.

For topical solutions, a preferable concentration of drug is 0.05 to 10%. More preferable is 0.1 to 5%. Most preferable is 0.1 to 2%. A preferable ratio of lipid/or polymer-lipid to the drug (lipid/drug) is 1 to 30. More preferable is 1 to 20. Most preferable is 1 to 15.

For intra-mouth spray, a preferable concentration of drug is 0.05 to 5%. More preferable is 0.1 to 3%. Most preferable is 0.1% to 2%. A preferable ratio of lipid/or polymer-lipid to the drug (lipid/drug) is 1 to 20. More preferable is 1 to 15. Most preferable is 1 to 10.

For intranasal spray, a preferable concentration of drug is 0.05 to 5%. More preferable is 0.1 to 5%. Most preferable is 0.1% to 2%. A preferable ratio of lipid/or polymer-lipid to the drug (lipid/drug) is 1 to 20. More preferable is 1 to 15. Most preferable is 1 to 10.

For gel preparation, a preferable concentration of drug is 0.05 to 5%. More preferable is 0.1 to 3%. Most preferable is 0.1% to 2%.

For ointment preparations, a preferable concentration of active is 0.5 to 10%, more preferable is 0.1 to 5%, most preferable is 0.1 to 2%. A preferable ratio of lipid/or polymer-lipid to the drug (lipid/drug) is 1 to 30, more preferable is 1 to 20, most preferable is 1 to 15.

### EXAMPLE 1

### In Vitro Activity Test

Organisms listed in Table 1 were tested according to an agar dilution method: Suspensions of each microorganism were prepared to contain 105 colony forming units (cfu)/mL. All drugs were dissolved in a few drops of DMSO then diluted with ethanol—water (1/1, v/v) to make a stock solution of 500 μg/mL. The agar dilution method was performed in a medium of Kimmig's agar (K. A., Merck) −0.5% glycerol [R. A. Fromtling, G. K. Abruzzo and A. Ruiz, Mycopathologia, 106 (1989) 163-166]. Plates of Kimmig's agar containing serial dilutions (25 to 0.01 μg/mL) of the drugs were inoculated with 10 μL of the fungal inocula and incubated at 25° C. during days for yeasts and up to 5 days for filamentous fungi. Following incubation, GMMICs (geometric mean minimum inhibitory concentration μg/mL) were determined. The results are shown in Table 2. In the table POCZ indicates posaconazole, ITRZ indicates itraconazole, EQUZ indicates Equaconazole, and FLUZ indicates fluconazole [Patterson, T. F., S. G. Revankar, W. R. Kirkpatrick, O. Dib, A. W. Fothergill, S. W. Redding, D. A. Sutton, and M. G. Rinaldi, J. Clin. Microbiol. 34 (1996) 1794-1797].

TABLE 1

| | | GMMICS (μg/mL) | | | |
|---|---|---|---|---|---|
| Organism | No. Organism | EQUZ | POCZ | ITRZ | FLUZ |
| Aspergillus Flavus | 9 | 0.09 | 0.12 | 0.35 | >235 |
| Candida Krusei | 22 | 0.15 | 0.21 | 0.60 | 65 |
| Cryptococcus | 10 | 0.25 | 0.24 | 0.49 | 45 |
| neoformans | | | | | |
| Trichophyton | 17 | 0.10 | 1.2 | 3.1 | 105 |
| rubrum | | | | | |
| Microsporum canis | 6 | 0.35 | 0.50 | 1.2 | 151 |

Compounds of the invention were tested for their ability to inhibit ergosterol biosynthesis. Testing was performed in 96-well round-bottom microtitration plates (Table 2). Cell suspensions were prepared in RPMI-1640 medium and were adjusted to give a final inoculum concentration of 0.5×10³ to 2.5×10³ cells/ml. The plates were incubated incubated at 30° C. for 48 h before growth was assessed. The MIC of each compounds was defined as the lowest concentration at which there was 80% inhibition of growth compared with that in a drug-free control [O. N. Breivik and J. L. Owades, Agric. Food Chem., 5 (1957) 360-363; T. F. Patterson, S. G. Revankar, W. R. Kirkpatrick, O. Dib, A. W. Fothergill, S. W. Redding, D. A. Sutton and M. G. Rinaldi, J. Clin. Microbiol., 34 (1996) 1794-1797]. Ergosterol content was calculated as a percentage of the wet weight of the cell [National Committee for Clinical Laboratory Standards. 1997. Reference method for broth dilution antifungal susceptibility testing of yeasts, Approved standard. Document M27-A, National Committee for Clinical Laboratory Standards, Wayne, Pa.].

TABLE 2
IC50 Values for inhibition of Ergosterol Biosynthesis

| | |
|---|---|
| Candida | Aspergillus |
| albicans_C286 | fumigatus_ND 158 |

| Agent | (nM) | Agent | (nM) |
|---|---|---|---|
| ITRZ | 39.1 | POCZ | 11.8 |
| EQUZ | 31.1 | EQUZ | 9.8 |

These in vitro studies demonstrated favorable or comparable activity for Equaconazole when compared to posaconazole, itraconazole, and fluconazole against a variety of fungal pathogens. Furthermore, equaconazole was shown to demonstrate favorable antifungal activity against Ergosterol Biosynthesis.

### EXAMPLE 2

### PK Study of Antifungal Nasal Spray

Experiments were performed to determine blood levels of Posaconazole formulation after intranasal dosing in Gottingen mini-pigs. A Group of three male and three female mini-pigs (ages from 6 to 9 months) were used for the studies. The vehicle used in the experiments was 5% of PEG-12 GDM in saline solution. The intranasal administration was performed with 35 mL of Luer slip syringes. HPLC-MS analyses were performed on heparinized mini-pig plasma samples obtained typically at 0 hr, 0.08 hr, 1 hr, 2 hr, 4 hr, 12 hr and 24 hr after dosing. The drug was first isolated from plasma with a sample pre-treatment. Acetonitrile were used to remove proteins in samples. An isocratic HPLC-MS method was then used to separate the drug from any potential interference. Drug levels were measured by MS detection with a multiple reaction monitoring (MRM) mode. Only trace amounts (Table 3) of Posaconazole were detected in the mini-pig plasma after dosing with the formulated Posaconazole. The results demonstrated that nasal administration of the triazole drug had negligible entry into the blood circulation of the animals.

TABLE 3
Posaconazole Level in Mini-pig Plasma

| Time | Posaconazole (ng/mL) | | | | | |
|---|---|---|---|---|---|---|
| (hour) | Male 1 | Male 2 | Male 3 | Female 1 | Female 2 | Female 3 |
| 0 | ND* | ND | ND | ND | ND | ND |
| 0.08 | 1.35 | 1.74 | 1.56 | 1.23 | 1.39 | 1.21 |
| 1 | 1.62 | 1.24 | 1.26 | 2.06 | 1.86 | 1.26 |
| 4 | 1.26 | 1.42 | 1.46 | 1.28 | 1.17 | 1.56 |
| 12 | 1.96 | 2.41 | 1.93 | 2.03 | 2.44 | 2.35 |
| 24 | 1.86 | 1.86 | 1.66 | 1.17 | 2.04 | 1.46 |

*ND = Not detected

### EXAMPLE 3

### Triazole Nebulizer Solution

A lipid based formulation suitable for nasal delivery of triazoles was prepared. PEG-Lipid or other solubilizer(s) was added to a vessel equipped with a mixer propeller. The drug substance was added with constant mixing. Mixing continued until the drug was visually dispersed in the lipids. Pre-dissolved excipients in water were slowly added to the vessel with adequate mixing. Mixing continued until fully a homogenous solution was achieved. A sample formulation is described in Tables 4 and 5.

TABLE 4

| Ingredient | mg/g |
|---|---|
| Active ingredient | 5 |
| Carboxymethylcellulose sodium | 15 |
| Dextrose | 50 |
| Benzalkonium chloride | 0.1 |
| Phenylethyl alcohol | 0.003 |
| Polysorbate 80 | 0.05 |
| HCl | To adjust pH 6 to 7 |
| Purified water | qs to 1,000 mg |

TABLE 5

| Ingredient | mg/mL |
|---|---|
| Active ingredient | 5 |
| Solublizer | 20 |
| Sodium Chloride | 9 |
| Lactic acid | 10 |
| Sodium Hydroxide | See below |
| Benzalkonium Chloride | 0.1 |
| Purified Water | qs 1 mL |

The active ingredient may be Posaconazole or Equaconazole or Voriconazole. The PEG-lipid or solublizer may be DAG-PEG or LCP or fatty alcohol ethoxylate or fatty acid ethoxylate or ethoxylated glycol and glycerol esters or ethoxylated natural oils and fats or any commercially available formulated O/W emulsifiers, or any combinations thereof. Sodium hydroxide was used to prepare a 10% w/w solution in purified water. The NaOH solution is used to adjust pH and the targeted pH was in a range of 4.0 to 7.0. The drug to lipid ratio is preferably greater than about 1 to 20, and more preferably greater than about 1 to 5. Other organic acid, pyruvic acid or glycolic acid, may be used though lactic acid is most preferable. The concentration of organic acid is preferably in the range 1 and 10%, and more preferably about 2 to 5%.

### EXAMPLE 4

### Triazole Topical Cream

PEG-lipid was added to a stainless steel vessel equipped with propeller type mixing blades. The drug substance was added with constant mixing. Mixing continued until the drug was visually dispersed in the lipids at a temperature to 60°-65° C. Organic acid, Cholesterol and glycerin were added with mixing. Ethanol and ethyoxydiglycol were added with mixing. Finally Carbopol ETD 2020, purified water and triethylamine were added with mixing. Mixing continued until fully a homogenous cream was achieved. The formulation is described in Table 6.

TABLE 6

| Ingredient | % |
|---|---|
| Active ingredient | 1.0 |
| PEG Lipid | 5.0 |
| Carbopol ETD 2020 | 0.5 |
| Ethyoxydiglycol | 1.0 |
| Ethanol | 5.0 |
| Glycerin | 1.0 |
| Cholesterol | 0.4 |
| Triethylamine | 0.20 |
| Organic acid | 5 |
| Sodium hydroxide | See below |
| Purified water | qs 100 |

The active ingredient may be Posaconazole or Equaconazole or Voriconazole. The lipid may be DAG-PEG or a LCP or other solubilizers or combination thereof (see Example 3). Organic acid may be lactic acid or pyruvic acid or glycolic acid. Sodium hydroxide was used to adjust pH if necessary. The targeted pH range was between 3.5 and 7.0.

### EXAMPLE 5

### Triazole Topical Cream

The topical cream was prepared similarly as described in Example 4 with different the excipients. A sample formulation is described in Table 7.

TABLE 7

| Ingredient | % |
|---|---|
| Active ingredient | 5 |
| Propylene Glycol | 100 |
| Sodium Phosphate, monobasic | 2.7 |
| Phosphoric acid | 0.03 |
| White Petrolatum | 150 |
| Mineral Oil | 50 |
| Ceteareth-30 | 20 |
| Cetostearyl Alcohol | 80 |
| 4-chloro-m-cresol | 1 |
| Purified water | Qs to 1,000 mg |

The active ingredient may be Posaconazole or Equaconazole or Voriconazole.

### EXAMPLE 6

### Triazole Topical Solution

The topical solution was prepared as in Example 3, except that active was first dissolved in organic acid and ethanol. A sample formulation is described in Table 8.

TABLE 8

| Ingredient | % |
|---|---|
| Active ingredient | 1.0 |
| PEG Lipid | 5.0 |
| α-Tocopherol | 0.5 |
| Lactic acid | 2.5 |
| Ethanol | 5.0 |
| Sodium benzoate | 0.2 |
| Sodium hydroxide | See Below |
| Purified Water | qs 100 |

The active ingredient may be Posaconazole or Equaconazole or Voriconazole. The PEG-lipid may be a DAG-PEG or LCP or fatty alcohol ethoxylate or fatty acid ethoxylate or ethoxylated glycol and glycerol esters or ethoxylated natural oils and fats or any commercially available formulated O/W emulsifiers, or any combinations thereof. Sodium hydroxide was used to prepare a 10% w/w solution in purified water. The NaOH solution is used to adjust pH and the targeted pH was in a range of 4.0 to 7.0. The drug to lipid ratio is preferably greater than about 1 to 20, and more preferably greater than about 1 to 5. Other organic acid, pyruvic acid or glycolic acid, may be used though lactic acid is most preferable. The concentration of organic acid is preferably in the range 1 and 10%, and more preferably about 2 to 5%.

### EXAMPLE 7

### Triazole Lotion

The lotion was prepared similarly as described in Example 4 except the excipients were different. A sample formulation is described in Table 9.

TABLE 9

| Ingredient | mg/mL |
|---|---|
| Active ingredient | 10 |
| White petrolatum | 50 |
| Ceteareth 30 | 20 |
| Cetearyl alcohol | 25 |
| Mineral oil | 20 |
| Phosphoric acid | 0.03 |
| Propylene glycol | 100 |
| Sodium hydroxide | See below |
| Sodium Phosphate, monobasic | 2.7 |
| Benzyl alcohol | 10 |
| Purified Water | Qs to 1,000 |

The active ingredient may be Posaconazole or Equaconazole or Voriconazole. The PEG-lipid may be a DAG-PEG or LCP or fatty alcohol ethoxylate or fatty acid ethoxylate or ethoxylated glycol and glycerol esters or ethoxylated natural oils and fats or any commercially available formulated O/W emulsifiers, or any combinations thereof. Sodium hydroxide was used to prepare a 10% w/w solution in purified water. The NaOH solution is used to adjust pH and the targeted pH was in a range of 4.0 to 7.0. The drug to lipid ratio is preferably greater than about 1 to 20, and more preferably greater than about 1 to 5. Other organic acid, pyruvic acid or glycolic acid, may be used though lactic acid is most preferable. The concentration of organic acid is preferably in the range 1 and 10%, and more preferably about 2 to 5%.

### EXAMPLE 8

### Triazole Solution

The nonaqueous solution was prepared with polyethylene glycol 400 or PEG-lipid. A sample formulation is described in Table 10 and Table 11.

TABLE 10

| Ingredient | % |
|---|---|
| Active ingredient | 1.0 |
| PEG-400 | 99 |

The active ingredient may be Posaconazole or Equaconazole or Voriconazole.

TABLE 11

| Ingredient | % |
|---|---|
| Active ingredient | 1.0 |
| PEG-lipid | 99 |

The active ingredient may be Posaconazole or Equaconazole or Voriconazole. The PEG-lipid may be DAG-PEG or LCP or fatty alcohol ethoxylate or fatty acid ethoxylate or ethoxylated ethoxylates glycol and glycerol esters or ethoxylated natural oils and fats or any combinations thereof.

### EXAMPLE 9

### Triazole Ointment

Each gram of the triazole ointment 1% contains 10 mg of the active ingredient in an ointment base of mineral oil and white petrolatum. A sample formulation is described in Table 12.

TABLE 12

| Ingredient | % |
|---|---|
| Active ingredient | 1.0 |
| Mineral oil | 5.0 |
| White petrolatum | 95 |

The active ingredient may be Posaconazole or Equaconazole or Voriconazole.

### EXAMPLE 10

### Triazole Gel

A clear, translucent gel for topical application was prepared as described in Table 13.

TABLE 13

| Ingredient | % |
|---|---|
| Active ingredient | 1.0 |
| Propylene Glycol | 70 |
| Carbomer 940 | 2 |
| Phosphoric acid | See below |
| Sodium hydroxide | See below |
| Purified water | qs 100 |

The active ingredient may be Posaconazole or Equaconazole or Voriconazole. Sodium hydroxide was used to prepare a 10% w/w solution in purified water. The NaOH solution is used to adjust pH with phosphoric acid and the targeted pH was in a range of 4.0 to 6.0.

### EXAMPLE 11

### Triazole Aerosol

Triglyceride, triazole and mineral oil were mixed in a jacked stainless steel vessel at 50-55° C. to dissolve the drug. The mixture was cooled to room temperature with agitation. The uniform solution was filtered into a clean container. The concentrated solution was then delivered with isopropyl alcohol into each can and filled with a hydrocarbon propellant (i.e., a mixture of propane and isobutane). A sample formulation is described in Table 14.

TABLE 14

| Ingredient | %/can |
|---|---|
| Active ingredient | 0.2 |
| Caprylic/capric triglyceride | 4.5 |
| mineral oil | 1.5 |
| Isopropyl alcohol | 9.9 |
| Propellent | 84 |

The active ingredient may be Posaconazole or Equaconazole or Voriconazole.

### EXAMPLE 12

### Triazole Intra-Mouth Spray Solution

The intra-mouth spray solution was prepared as in Example 3, except that active was first dissolved in organic acid and ethanol. A sample formulation is described in Table 15.

TABLE 15

| Ingredient | mg/mL |
|---|---|
| Active ingredient | 5 |
| Solublizer | 20 |
| Sodium Chloride | 9 |
| Lactic acid | 10 |
| Sodium Hydroxide | See below |
| Benzalkonium Chloride | 0.02 |
| Ethanol | 20 |
| Purified Water | qs 1 mL |

The active ingredient may be Posaconazole or Equaconazole or Voriconazole. The solublizer may be DAG-PEG or LCP or fatty alcohol ethoxylate or fatty acid ethoxylates or ethoxylated glycol and glycerol esters or ethoxylated natural oils and fats or any commercially available formulated O/W emulsifiers, or any combinations thereof. Sodium hydroxide was used to prepare a 10% w/w solution in purified water. The NaOH solution is used to adjust pH and the targeted pH was in a range of 4.0 to 7.0. The drug to lipid ratio is preferably greater than about 1 to 20, and more preferably greater than about 1 to 5. Other organic acid, pyruvic acid or glycolic acid, may be used though lactic acid is most preferable. The concentration of organic acid is preferably in the range 1 and 10%, and more preferably about 2 to 5%.

While preferred embodiments of the present invention have been described, those skilled in the art will recognize that other and further changes and modifications can be made without departing from the spirit of the invention, and all such changes and modifications should be understood to fall within the scope of the invention.

We claim:

1. A pharmaceutical composition comprising:
a pharmaceutically effective amount of a triazole including at least one of Posaconazole, Equaconazole, and Voriconazole; and
a carrier vehicle for topical use, said carrier vehicle comprising one or more solubility or permeability enhancers.

2. The pharmaceutical composition according to claim 1 wherein the solubility or permeability enhancers are selected from the group consisting of diacylglycerol-polyethyleneglycol, sugar-lipid-polymer, fatty alcohol ethoxylates, fatty acid ethoxylates, ethoxylated glycol, glycerol esters, ethoxylated natural oils and fats, commercially available formulated oil/water emulsifiers, and combinations thereof.

3. The pharmaceutical composition of claim 1 comprising the triazole at a concentration in the range of 0.01-10%.

4. The pharmaceutical composition of claim 1 wherein the solubility or permeability enhancers comprise lipid, lipids, or polymer modified lipids.

5. The pharmaceutical composition of claim 4 comprising the solubility or permeability enhancers at a concentration in the range of 0.01-99.09%.

6. The pharmaceutical composition of claim 1 wherein the composition is a solution.

7. The pharmaceutical composition of claim 1 wherein the composition is a gel.

8. The pharmaceutical composition of claim 1 wherein the composition is a lotion.

9. The pharmaceutical composition of claim 1 wherein the composition is a cream.

10. The pharmaceutical composition of claim 1 wherein the composition is an ointment.

11. The pharmaceutical composition of claim 1 wherein the composition is a nasal spray.

12. The pharmaceutical composition of claim 1 wherein the composition is an intra-mouth spray.

13. The pharmaceutical composition of claim 1 wherein the composition is an aerosol.

14. A method for the treatment or prevention of fungal infections with the pharmaceutical composition of claim 1 comprising the step of administering, to a subject, the pharmaceutical composition in an amount sufficient for treating or preventing fungal infection.

15. The method for the treatment or prevention of fungal infections of claim 14 wherein the administering step comprises administering the composition for the treatment of feet, hands, nails, skin, mucus, or combinations thereof.

16. The method for the treatment or prevention of fungal infections claim 14 wherein the administering step comprises topically applying the pharmaceutical composition.

17. The method for the treatment or prevention of fungal infections claim 14 wherein the administering step comprises administering the composition for the treatment of dermal tissue, subdermal tissue, mucosal membranes, or combinations thereof.

18. The method for the treatment or prevention of fungal infections claim 14 wherein the administering step comprises administering the composition for the treatment of at least one of tinea pedis, tinea manuum, tinea unguium, tinea cruris, tinea corpora, tinea versicolor, candidiasis, aspergillosis, onychomycosis, coccidiodomycosis, cryptococcal meningitis, histoplasmosis, hoof thrush, hoof rot, tongue and mouth lesions, and seborrheic dermatitis.
